(19) United States
(12) Patent Application Publication
(10) Pub. No.: US 2022/0249275 A1
(43) Pub. Date: Aug. 11, 2022

(54) IUD INSERTION DEVICE

(71) Applicants: Michael Lynn STITELY, Dunedin (NZ); Gwynneth Helen PATERSON, Dunedin (NZ); Stuart MAY, Rozelle, New South Wales (AU); Robert John SMITH, Rozelle, New South Wales (AU); Greta Isabella SAGGUS, Rozelle, New South Wales (AU); Otago Innovation Limited, Dunedin (NZ)

(72) Inventors: Michael Lynn STITELY, Dunedin (NZ); Gwynneth Helen PATERSON, Dunedin (NZ); Stuart MAY, Rozelle (AU); Robert John SMITH, Rozelle (AU); Greta Isabella SAGGUS, Rozelle (AU)

(21) Appl. No.: 17/628,527

(22) PCT Filed: Jul. 17, 2020
(86) PCT No.: PCT/NZ2020/050069

§ 371 Date: Jan. 19, 2022

(30) Foreign Application Priority Data
Jul. 19, 2019 (NZ) 755578

(51) Int. Cl. A61F 6/18; A61F 6/14

(52) U.S. Cl. CPC A61F 6/18; A61F 6/144

(57) ABSTRACT

An insertion device for an IUD. The insertion device comprises an introducer having an elongate hollow body and a first end and a second end, the hollow body defining an elongate passageway extending from the first end to the second end and defining openings at the first end and the second end, and a pusher provided or configured to be provided inside the elongate passageway such that the pusher is movable through or along the passageway. The passageway is sized to accommodate an IUD such that the IUD is slidable along the passageway. Further, the second end of the introducer that abuts the fundus of the uterus in use is configured to have an increased surface area relative to prior art devices so as to reduce the risk of injury, including perforation of the uterus.

### FIELD

The disclosure relates to insertion devices for intrauterine devices (IUDs), particularly those for insertion of IUDs in postpartum women.

### BACKGROUND

Motivation to initiate contraceptive use postpartum is high. The World Health Organisation considers that up to 100,000 maternal deaths per year could be avoided if effective contraception was used by women that did not want children. Further, preventing unintended pregnancies could avert the loss of 4.5 million disability-adjusted life years.

IUDs are an effective form of long-acting, reversible contraception. They are often a generally T-shaped structure and there are two basic variants, copper and hormonal. An example of a hormonal IUD is shown in FIG. 1 and is taken from the Medsafe data sheet for the Mirena® 52 mg IUD. The ParaGard® is an example of a copper IUD, further details of which are provided later.

FIG. 2 shows the IUD of FIG. 1 in position inside the uterus. String is attached to the base of the stem of the T and hangs down inside the uterus. The string is gripped and pulled to remove the IUD. The arms are flexible to allow the device to become more compact for ease of movement of the IUD into and out of the uterus, in particular during passage through the cervix. The arms ultimately extend laterally when the IUD is fully inserted up to the fundus or top of the uterus and hold the IUD in position.

The inventor has recognised that postpartum insertion of IUDs could be an effective way of reducing unwanted pregnancies but there are obstacles limiting wider uptake. Current methods of postpartum IUD (PPIUD) insertion are difficult to learn for non-obstetrician birth attendants such as midwives and GPs. One survey in the US found that only 10% of midwives feel confident inserting a PPIUD and 90.7% of American midwives had never inserted a PPIUD.

IUDs are traditionally inserted using forceps. Essentially, the IUD is gripped with the forceps and then inserted through the cervix and up to the fundus. More particularly, the following steps may be taken for postpartum or postplacental insertion of IUDs:
1. Cleanse the cervix and vagina with betadine (antiseptic) gripping the stem of the T with forceps and change into new sterile gloves.
2. Grasp the anterior cervical lip with ring forceps.
3. Grip the IUD with ring forceps.
4. Gently pull the cervix-holding forceps and insert the IUD-holding forceps through the cervix and into the lower uterine cavity, trying to avoid touching of the walls of the uterus.
5. Release the hand used to grip the cervix and place the hand on the abdomen, palpating the fundus. With the same hand, stabilise the uterus with firm downward pressure.
6. Move the IUD-holding forceps to the fundus and open the forceps to release the IUD.
7. Remove the forceps.

To simplify this process, IUD insertion devices have been devised.

FIG. 3 shows an example copper IUD, namely, the Multiload. As can be seen, the arms are bent somewhat, deviating slightly from the more traditional T-shapes, and projections extend out laterally from the arms. The trunk of the device is formed from a plastic rod with copper wire wound thereabout. Some copper IUDs also include copper provided to the laterally extending arms.

Reference is now made to the New Zealand data sheet, Version 4.0 prepared November 2012, for the Multiload device. Relevant parts of the insertion procedure for non-postpartum women detailed in this document are repeated below.
a. Insert a vaginal speculum to expose the cervix. Cleanse the cervix and vaginal walls with sterile cotton wool dipped in antiseptic solution. Wipe all secretion away from the external os.
b. Grasp the anterior lip of the cervix with a single-tooth tenaculum, taking a good bite through the cervical lip so that steady downward traction to straighten the uterine axis can be maintained without risk of cervical laceration. Reflex contraction, which causes cramp of the uterus when the tenaculum is applied, can be prevented by injection of a local anaesthetic into the anterior lip or a paracervical block.
c. Carefully sound the uterus to determine its depth and to confirm the direction of its axis. If the sound meets more than normal resistance at the internal os, it may be advisable to gently dilate the cervical canal to 4-5 mm, using sterile, tapered rather than cylindrical dilators. In the absence of other instruments for measurement of the internal dimensions of the uterine cavity, the sound may be used to obtain an idea of its configuration.

As will be apparent, steps (a)-(c) are essentially the same as steps (1)-(3) of the forceps-based method. However, subsequent steps are modified when using the insertion device. The insertion device is essentially a narrow tube (referred to as the “introducer tube”) that houses the trunk and tips of the arms of the IUD during insertion.
d. The vertical stem of MULTILOAD is already preloaded in the introducer tube. The side arms do not require loading into the tube. They are sufficiently flexible to adapt to the shape of the cervical canal. Tear the pouch open at the indicated notch.
e. Peel the clear cover back so far that the introducer tube (with IUD) can be picked up at its distal end, grasping the tube and the threads, but without taking MULTILOAD out of the pouch.
f. Hold the cervical stop with the thumb of one hand and adjust the position of the top of MULTILOAD by moving the introducer tube with the other hand until it corresponds with the mark indicating, approximately, the sounded uterine length in centimetres.
g. The distal end of the introducer may be held without risk of contaminating the device. Holding the threads together with the tube ensures that the device does not fall out of the introducer tube. MULTILOAD can now be taken out of the pouch.
h. Carefully insert MULTILOAD into the uterus (see FIG. 4) until it touches the fundus and the cervical stop rests against the external os (see FIG. 5) while maintaining steady downward traction with the tenaculum to straighten the uterine axis. No attempt should be made to force insertion. Insufficient axial straightening may, on occasion, result in a sub-endometrial insertion. This risk may be reduced by exerting an adequate downward pulling force on the cervix, thereby fully straightening the axis of the uterus against its ligamentous supports.
i. When MULTILOAD touches the fundus, it is released into the uterine cavity by simply withdrawing the introducer tube (see FIG. 6). During this procedure continue to apply downward traction with the tenaculum. No push-rod is required to insert MULTILOAD. Check the cervical canal with the sound to ensure that the tail of MULTILOAD is entirely within the uterine cavity. Trim the threads of MULTILOAD to 2 to 3 cm measured from the external os.
j. It is imperative to follow precisely the recommended insertion procedure in order to minimise the risk of a sub-endometrial insertion, which may, in turn, lead to full or partial endometrial embedding of the IUD. Should this occur, a higher than normal force may need to be applied to remove the IUD from this incorrect location, which may increase the risk of side-arm breakages. Furthermore, it may be clinically difficult to confirm the IUD's sub-endometrial location, since this is usually not obvious to the doctor during insertion of the device and the patient probably experiences no pain. It is anticipated that correctly inserting the device may reduce the incidence of both side-arm breakages and perforations.

This method may be modified to the following for postpartum women:
i. Insert a bivalve (bayonet) speculum to expose the cervix after delivery of the placenta and membranes (no later than 10 minutes). Cleanse the cervix and vaginal walls with sterile cotton wool dipped in antiseptic solution.
ii. Grasp both the anterior and the posterior lips with one or two ring forceps and draw the cervix down for close inspection.
iii. Take the introducer tube (with pre-loaded IUD) and insert MULTILOAD along the palmar aspect of two fingers into the uterine cavity until it touches the fundus. Check the position of MULTILOAD with the flat hand on the abdominal wall covering the fundal region.
iv. When MULTILOAD touches the fundus, it is released into the uterine cavity by very gentle withdrawal of the introducer tube. Take care not to pull on the threads which are left uncut until the first follow-up visit.

The Paragard® is also provided with an introducer tube but further includes a push rod which can serve to properly locate the IUD at the fundus. Instructions for insertion of the Paragard® T380A are provided in the relevant FDA data sheet. FIGS. 7-13 accompanying this specification are FIGS. 1-7 from this document and show the step-by-step process.

In short, the packaging is opened (FIG. 7), the arms of the IUD are folded together and inserted into the introducer tube (FIG. 8), with some part of the trunk and arms of the IUD still extending beyond the introducer tube. The introducer tube and IUD are then inserted so that the tip abuts the fundus (FIG. 9) and then, with the push rod held in position, the introducer tube is removed a small distance to release the arms of the IUD from the confines of the introducer tube (FIG. 10). The introducer tube is then pushed upwards to ensure full insertion of the device (FIG. 11) and with the introducer tube held in position, the push rod is removed (FIG. 12). The introducer tube is then removed, leaving the IUD in position (FIG. 13).

Such insertion devices have been modified for use postpartum but the changes are fundamentally to the length of the device—due to enlargement of the uterus, the distance to the fundus is greater, so the introducer tube, push rod and strings for removal are longer (see Comparative safety and efficacy of a dedicated postpartum IUD inserter versus forceps for immediate postpartum IUD insertion: a randomized trial, Contraception 98 (2018), 215-219, Paul D. Blumenthal, Klaira Lerma, Renita Bhamrah, Sharad Singh).

As will be apparent, postpartum, the uterus is healing and is particularly susceptible to injury albeit perforation rates are low and comparable between postpartum and non-postpartum women. A. K. Whitaker, B. A. Chen, Society of Family Planning Guidelines: Postplacental insertion of intrauterine device, Contraception 97 (2018) 2-13 compared expulsion rates of IUDs in postpartum and non-postpartum women. The expulsion rates are generally higher in postpartum women and there is a belief this is because they are less likely to be inserted properly due to the inserter's anxieties of injuring a patient (including perforation of the uterus) during the procedure. Consequently, IUDs may not always be provided to the fundus.

Insertion devices to date have improved ease of use somewhat over forceps-based insertion methods by not requiring handling of the IUD with forceps but such devices still present a significant risk in damaging the uterus.

### SUMMARY OF THE INVENTION

According to a first aspect, there is provided an insertion device for an IUD, the insertion device comprising an introducer having an elongate hollow body and a first end and a second end, the hollow body defining an elongate passageway extending from the first end to the second end and defining openings at the first end and the second end, and a pusher or plunger provided or configured to be provided inside the elongate passageway such that the pusher is movable through or along the passageway, wherein the passageway is sized to accommodate an IUD such that the IUD is slidable along the passageway.

According to some preferred embodiments, a maximum straight line distance between two points on the exterior surface and at the second end of the introducer is greater than 1 cm, more preferably greater than 1.5 cm, more preferably greater than 2 cm, more preferably greater than 2.5 cm, more preferably between 2.5 cm and 4 cm, more preferably between 3 cm and 3.5 cm, and most preferably is about 3.4 cm.

At least a portion of the exterior surface of the introducer may be cylindrical. The at least a portion of the exterior surface that is cylindrical may be the entire exterior surface, may extend from the first end to a point distal from the first end and between the first and second ends, or may extend from the second end to a point distal from the second end and between the first and second ends. Further still, at least a portion of the exterior surface may form a cylinder that is located between the first and second ends but is spaced from the first and second ends.

The cylindrical portion may have a constant radius.

While generally circular profiles are preferable for the introducer so as to be received by the patient without undue discomfort, the disclosure is not limited thereto. For example, rounded or oval cross-sections are also possible. Further, the cross-section of the introducer may vary along at least part of its length between the first and second ends. However, sharp edges are to be avoided as they may cause injury. So other shapes that are generally rounded or have rounded edges may be used.

According to a preferred embodiment, the exterior surface of the introducer is generally bulbous or flares outwards at the second end. Providing a bulbous or similarly contoured surface at the second end of the introducer helps to remove or avoid sharp edges that can cause injury. Further, the bulbous formation may be configured to increase the surface area of the end wall at the second end resulting in an increased force required to cause injury to a patient. Non-bulbous arrangements are also possible but bulbous shapes are preferred as they assist in insertion and removal. For example, other generally rounded, flared configurations may be used such as circular or elliptical cones, truncated at the base to join the narrower cross-section part of the introducer. Again, non-round shapes but with rounded corners may be used. These still provide for less concentration of forces and hence less likelihood of injury than prior art devices but will tend to not do so to quite the same extent as more generally rounded shapes.

The introducer may comprise a grip portion integrally formed therewith or coupled thereto, preferably at or proximate to the first end. The grip portion may be formed by contouring of the outer surface of the introducer at or proximate to the first end.

The introducer may be configured such that at least a portion of the elongate passageway is circular. Additionally or alternatively, the introducer may be configured such that at least a portion of the elongate passageway is non-circular, such as oval.

According to a preferred embodiment, at least a portion of the elongate passageway extending from or proximate the first end is non-circular and transitions to being circular. The transition may be linear or non-linear.

A cross-sectional area of the space formed by the elongate passageway may transition from a first area at the first end to a second area at a point between the first end and the second end, wherein the first area is greater than the second area. This provides a sloped entrance to the opening at the first end, making it easier to insert an IUD from that end and to insert the pusher.

The transition may be linear or non-linear, including curved or arcuate.

A cross-sectional area of the space formed by the elongate passageway at the second end may be greater than a cross-sectional area of the space formed by the elongate passageway at a point between the first end and the second end.

A cross-sectional area of the space formed by the elongate passageway may transition from a third area at the second end to a fourth area at a point between the first end and the second end, wherein the third area is greater than the fourth area.

The transition may be linear or non-linear including curved or arcuate.

The second area may be the same as the fourth area.

The wall forming the elongate hollow body of the introducer may have a constant or substantially constant thickness.

The introducer may be formed from a plastic or a transparent plastic, or from a metal.

The insertion device may comprise one or more orientation features for orientating the pusher and/or the IUD inside the passageway. The orientation features may comprise one or more longitudinal grooves in the elongate passageway configured to receive a protrusion of the pusher and/or of the IUD. Additionally oe alternatively, the orientation features may comprise one or more longitudinal ribs in the elongate passageway configured to engage a protrusion or a recess of the pusher and/or of the IUD. The orientation features may aid in movement of the pusher inside the introducer and/or better ensure the IUD is received by the patient in the correct orientation, improving success rates of insertion.

The insertion device may comprise a bump joint formed between the elongate passageway and the pusher and/or the IUD, said bump temporarily fixing the pusher and/or the IUD in a predetermined position in the passageway until additional force is used to urge the pusher.

The insertion device preferably comprises a pusher configured to be slidably engaged and/or received in the elongate passageway, the pusher comprising a first end and a second end.

The cross-section of the pusher may be complementary to the cross-section of at least the narrowest parts of the elongate passageway. However, to prevent the string of the IUD from being captured between the pusher and the walls defining the passageway, a sufficient gap is provided between the engaging walls, rather than there being a snug, piston-like engagement between the two.

The pusher may comprise an elongate rod. At least a portion of the rod may be generally cylindrical.

At least a portion of the rod may have a non-round cross-section.

The pusher may be formed from plastics.

The pusher may comprise a rod, preferably a metal rod. Where the pusher a rod, a body (e.g. formed from plastics) may be provided at the first end of the rod with the body being configured to engage with an IUD, and/or a handle portion may be provided at or near the second end of the pusher. The handle portion may be integrally formed with the rod or attached or attachable thereto.

The pusher may comprise a recess in the end wall of the first end thereof, the recess configured to receive a base of a trunk of an IUD. This serves to locate the IUD relative to the pusher and the passageway, making urging of the IUD along the passageway using the pusher easier and more reliable.

The first end of the pusher may comprise a pair of notches on opposing sides of the pusher. While non-essential, these can provide a preferred location for the strings of the IUD and thereby help to orientate the IUD relative to the pusher and/or passageway.

A pair of prongs may extend longitudinally from the sides of the first end of the pusher, the prongs configured to receive and/or engage the trunk or stem of an IUD therebetween.

The pusher may narrow moving from a position at or near the first end thereof to a smaller cross-section at a point farther from the first end.

The pusher may have a cutout or depression that extends longitudinally from a position at or near the first end of the pusher to a position farther from the first end.

The pusher may comprise a longitudinal rib that protrudes in a direction substantially orthogonal to an orientation of the IUD inside the passageway in use. The rib may provide added stiffness along the length of the pusher.

The introducer may comprise a groove in the elongate passageway that is complementary to the longitudinal rib of the pusher such that the pusher is slidable relative to the introducer when the rib is engaged with the groove.

The pusher may comprise one or more indicia to indicate the extent to which the pusher is inserted into the introducer.

The pusher may comprise a mechanical stop configured to engage corresponding feature of the introducer to limit the extent of insertion of the pusher into the introducer. Preferably, the mechanical stop is configured such that when the pusher is fully inserted, the pusher does not extend beyond the second end of the introducer. More preferably, on full insertion, the pusher is located inside the passageway and away from the second end of the introducer. For example, the pusher may be recessed from the second of the introducer by about 0.5 cm when fully inserted. This prevents the smaller cross-sectional area pusher from causing injury, such as perforation of the uterus when fully inserted. Further, it helps to reduce the likelihood of the pusher pushing the IUD into the uterus in a harmful way. This advantage is maximised by appropriate configuration of the passageway at the second end of the introducer as disclosed elsewhere herein.

The mechanical stop may comprise one or more projections on the pusher. Such projection(s) may be integrally formed or coupled to the pusher.

One or more projections may be provided on the introducer that extend into the elongate passageway so as to control a position and/or orientation of the pusher and/or IUD in use. The one or more projections may comprise a pair of opposing projections. The one or more projections may be provided proximate to but spaced apart from the second end of the introducer.

One or more indicia may additionally or alternatively be provided to the introducer, preferably to or visible on the outer surface thereof. These may indicate or limit an extent to which the introducer has been inserted into the patient.

Additionally or alternatively, an indicator may indicate a rotational orientation of the insertion device. As will be appreciated, a rotational orientation of the IUD about the axis formed by the trunk is important in ensuring proper alignment of the IUD inside the uterus. Such an indicator can assist the individual inserting the IUD in ensuring the IUD is correctly orientated. For example, a line may be provided on the external surface of the introducer extending at least part way between the first end and the second end, wherein the introducer and/or pusher are configured to orientate the IUD in a predetermined orientation relative to the indicator. For example, the indicator may be aligned with the arms or be perpendicular thereto, in the latter case such that the indicator faces the individual inserting the device during insertion.

The inside walls of the introducer that define the elongate passageway may be configured so as to provide a preferred orientation of an IUD therein. For example, the passageway may be elliptical such that arms of the IUD tend to locate along the major axis of the ellipse. Similarly, grooves may be provided on the inner walls.

The insertion device is particularly adapted for use on postpartum women.

More generally, the disclosure relates to IUD devices that help reduce harm to the uterus during IUD insertion, particularly postpartum IUD insertion. This may include increasing a surface area of parts of the end of the insertion device that are brought into contact with the fundus. For example, the dimensions at that end of the introducer may be increased as disclosed previously. Additionally or alternatively, a thickness of the walls defining the opening at that end of the introducer may be increased, albeit the opening may be configured per the prior art. Additionally or alternatively, the end wall that defines the opening may comprise a lip or a brim which, for example, may be rounded or extend into or away from the opening. Additionally or alternatively, at least the end parts of the introducer that abut the fundus in use may be formed from a softer or more compressible material than the main parts of the introducer and pusher. Such features may be used in combination with those above or isolated from the requirement of particular dimensions per the first aspect.

According to a second aspect, there is provided a method of inserting an IUD, comprising inserting an IUD into the elongate passageway of the introducer of the insertion device of the first aspect at the first end thereof, inserting the pusher into the elongate passageway at the first end of the introducer and urging the pusher along the elongate passageway a first distance, inserting the insertion device into the patient such that the second end of the introducer abuts the fundus, urging the pusher further into the elongate passageway such that the IUD springs free of the introducer, retracting the pusher from the introducer, and retracting the introducer from the patient.

Note that the device may be supplied with an IUD pre-installed, in which case the first step may be omitted as part of the method of inserting.

### BRIEF DESCRIPTION OF THE DRAWINGS

These and other features, aspects, and advantages of the present disclosure will be described with respect to the following figures, which are intended to illustrate and not limit the preferred embodiments.

FIG. 1 shows a prior art hormonal IUD.

FIG. 2 shows the IUD of FIG. 1 properly located inside the uterus.

FIG. 3 shows another example of a prior art IUD.

FIGS. 4-6 show insertion of the IUD of FIG. 3 into the uterus.

FIGS. 7-13 show a sequence of steps in inserting a prior art copper IUD into the uterus.

FIG. 14 is a perspective view of an embodiment of an IUD insertion device according to the present disclosure, with the pusher fully, or near fully, inserted in the introducer.

FIG. 15 is a perspective view of the device of FIG. 14 but with part of the wall of the introducer removed to show additional detail.

FIG. 16 is a view similar to that of FIG. 14 but with the pusher removed from the introducer.

FIG. 17 is a perspective view of the pusher and introducer shown in FIGS. 14-16 placed side-by-side.

FIG. 18 is a view similar to that of FIG. 17 but with part of the wall of the introducer removed to show additional detail.

FIGS. 19-26 show an alternative embodiment.

FIGS. 27-34 show a further embodiment.

FIGS. 35A and 35B show the patient engaging ends of another embodiment of an insertion device with the top half of the introducer removed to show internal detail.

FIGS. 36 and 37 show further alternative embodiments using views similar to that of FIG. 35A.

FIG. 38A shows an alternative embodiment of a pusher for use with an IUD insertion device, with only the end proximate the patient in use shown, and FIG. 38B shows the pusher of FIG. 38A inside an introducer.

FIGS. 39 and 40 show further embodiments of an IUD insertion device with a views similar to that in FIG. 35A.

FIGS. 41-45 provide various views of another embodiment of an IUD insertion device.

FIGS. 46-52 provide various views of another embodiment of an IUD insertion device.

FIGS. 53-63 provide various views of another embodiment of an IUD insertion device.

FIGS. 64-76 provide various views of another embodiment of an IUD insertion device.

FIGS. 77 and 78 show further alternative embodiments.

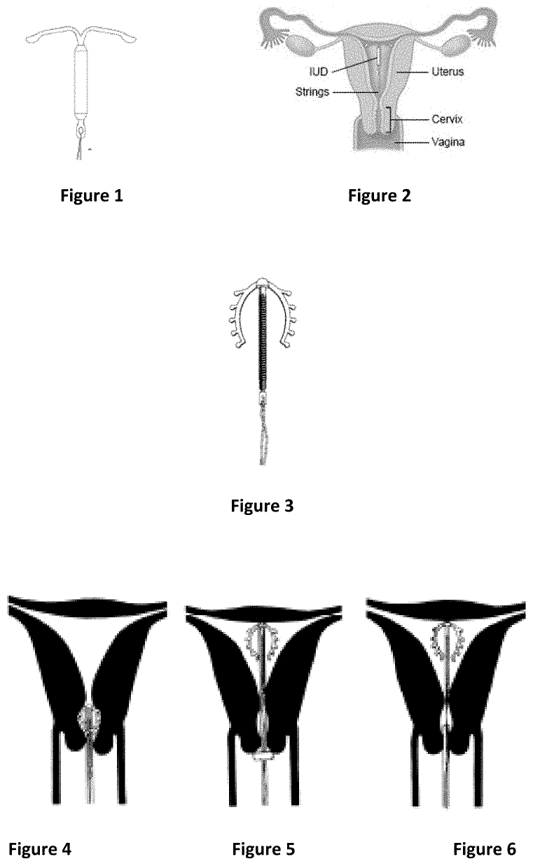

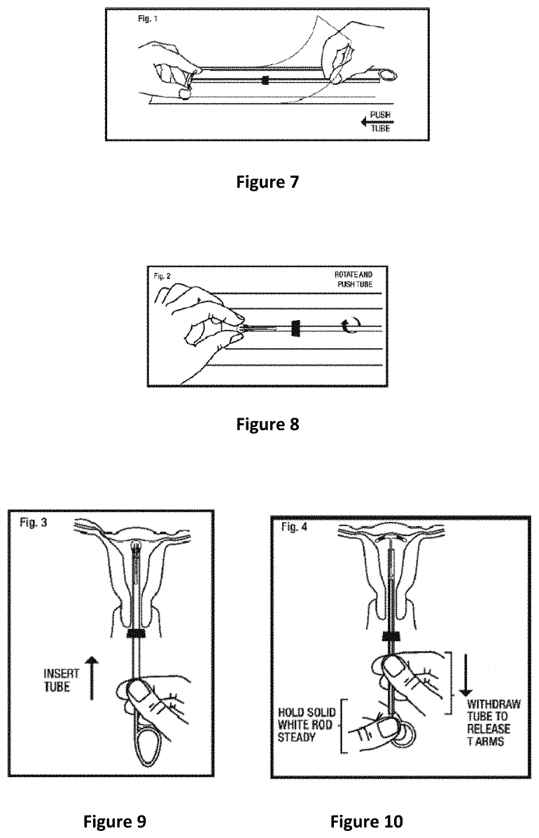

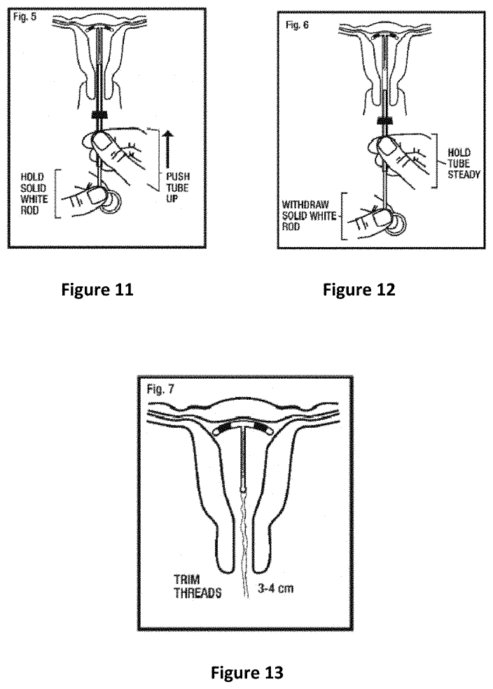

### DETAILED DESCRIPTION

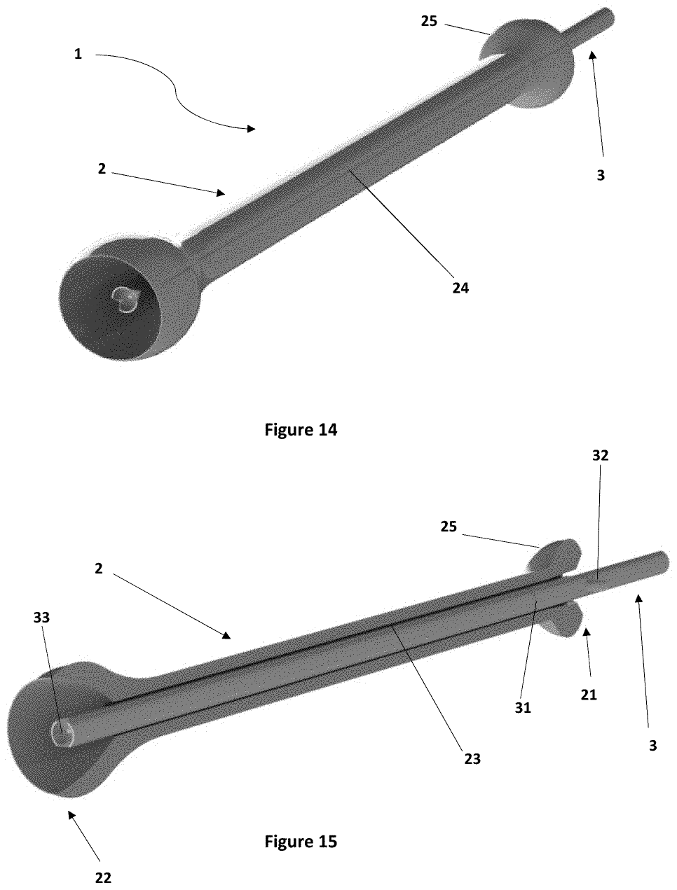

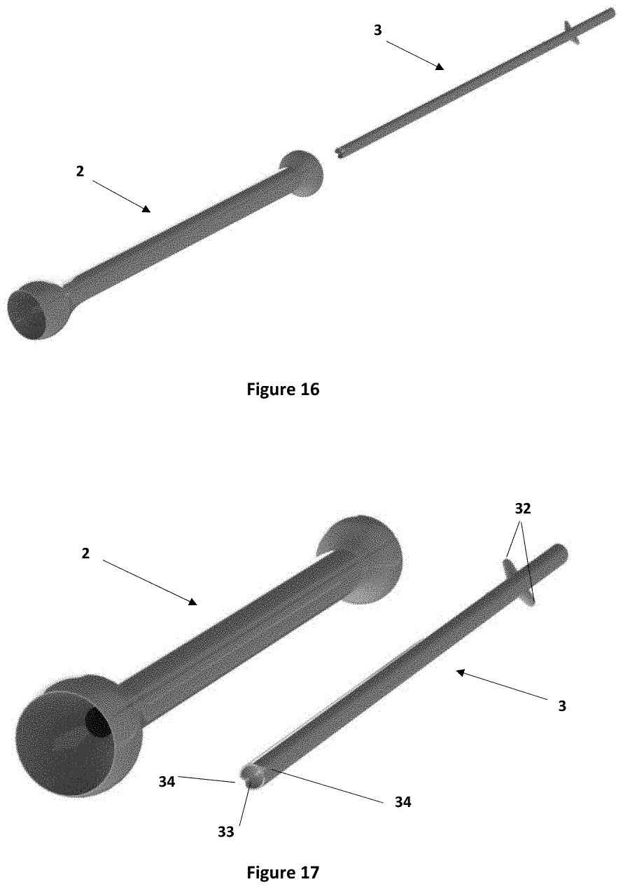

FIGS. 14-18 provide various views of an IUD insertion device 1 according to one embodiment. This IUD insertion device is particularly adapted for postpartum use.

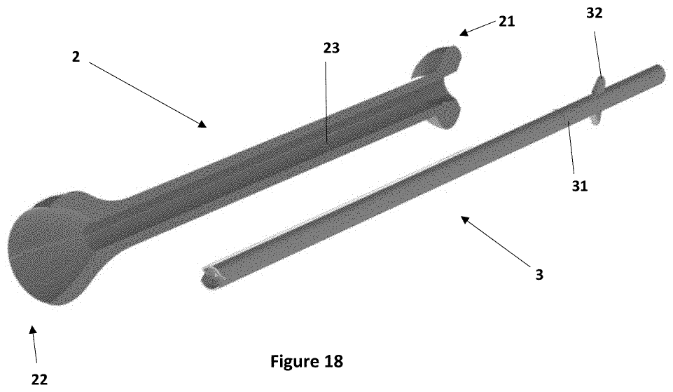

The insertion device 1 comprises an introducer 2 having an elongate hollow body and a first end 21 and a second end 22. The hollow body defines an elongate passageway 23 (best shown in FIG. 18) extending from the first end 21 to the second end 22, thereby defining openings at the first end and the second end of the introducer 2.

A pusher or plunger 3 is provided or configured to be provided inside the elongate passageway 23 such that the pusher 3 is movable through or along at least a portion of the passageway 23. The passageway 23 is sized to accommodate an IUD such that the IUD is slidable along the passageway 23.

While not limited thereto, an IUD may be inserted into the first end 21 of the introducer 2 with the end of the trunk of the IUD from which the arms extend leading the insertion. Inserting the IUD in this direction is preferable as it naturally encourages the arms of the IUD to fold on themselves in the desired manner such that they are aligned somewhat with the trunk. To more readily facilitate this, the opening at the first end 21 may be chamfered or angled such that the passageway 23 narrows from a first cross-sectional area at the first end 21 to a smaller cross-section part way between the first and second ends 21, 22 (see FIGS. 15 and 18). This transition region may be linear or non-linear, such as curved or arcuate, and guides the IUD, and the pusher 3, into the narrower part of the passageway 23.

According to a preferred embodiment, the passageway 23 has a portion extending from the first end 21 that is non-round. For example, it may be oval or grooves may be provided in the surface of the passageway along which the arms of the IUD locate and slide. The non-round shape of this portion of the passageway 23 can serve to rotationally orientate the IUD in the passageway 23. More particularly, the arms of the IUD will tend to locate themselves in the widest parts of the passageway 23. Having control of the rotational orientation of the IUD can better ensure that the IUD is received in the uterus in the correct orientation.

According to some embodiments, engaging features (not shown) are provided to or incorporated in the inner wall defining the passageway 23. These engaging features may be provided to releasably maintain an IUD in a preferred location in the elongate passageway 23. For example, one or more notches or grooves (including about part or an entire perimeter of the passageway 23) in which the ends of the arms of an IUD may sit in by inserting the IUD a first distance along the elongate passageway 23. Then, when the IUD is actually to be inserted, the pusher 3 may be urged further along the elongate passageway 23 whereby the arms of the IUD deform and disengage from the notches or grooves. The continued urging may be performed prior to insertion of the introducer 2 into position with the second end of the introducer 2 abutting the fundus of the uterus. Further, the insertion device 1 may be provided with an IUD already provided inside the elongate passageway 23, and preferably with the IUD located in this engaged position a first distance along the elongate passageway. The notches or grooves may be replaced with projections or ribs and operate in a similar manner, ensuring sufficient tolerance between the engaging walls of the introducer 2 and the pusher 3.

The introducer 2 may include an indicator to provide an indication of the orientation of the IUD inside the introducer 2. For example, as best shown in FIGS. 14 and 17, the introducer 2 may include a linear marking 24 along at least a portion of its exterior surface. This line may be in the form of a groove or a rib. The marking 24 may be aligned with the arms of the IUD inside the introducer 2, in which case two such markings may be provided on opposing sides of the introducer, or alternatively may be offset by 90 deg such that the marking 24 faces the medical professional during insertion. Again a pair of opposing such markings may be used, since, at least according to preferred embodiments, the introducer 2 has a longitudinal axis and is at least largely rotationally symmetrical about that axis except for any non-round portion of the passageway 23 the introducer 2, when provided, and the configuration of the grip portion 25. In any event, where this portion of the passageway 23 is non-round, it is preferably symmetrical about at least one plane through the axis. For example, where this portion of the passageway 23 is oval, it may be symmetrical about planes through the major and minor axes of the oval.

Prior art IUD insertion devices all have a very small cross-sectional area at the end thereof that is pressed towards the fundus of the uterus. Due to this very small area, relatively small forces exerted by medical personnel during insertion can result in injury, including puncturing of the uterus, as these forces are translated into considerable pressures. Postpartum women may be particularly vulnerable to injury because the immediate post-partum uterus is often relaxed prior to regaining full muscle tone following delivery. The “atonic” uterus feels soft to palpation, compared to a fully contracted post-partum uterus. Whilst the atonic uterus is most amenable to accessing the uterine cavity for intrauterine manipulations (such as inserting an IUD) this atony also places the postpartum uterus at risk for perforation due to the reduction in density of the muscle tissue in the uterus.

The inventor has recognised that increasing the surface area of the end wall at the second end 22 of the introducer 2 can reduce the pressure exerted by an IUD insertion device on the wall of the uterus, particularly the fundus. A preferred embodiment for realizing this is shown in FIGS. 14-18 but other designs are possible as will become apparent.

According to preferred embodiments, the second end 22 of the introducer 2 has a relatively large span. The increase in the size of the opening at the second end 22 results in a longer wall being required to define the opening and thus the surface area of the end wall at the second end 22 of the introducer 2 is larger than in prior art arrangements. According to some embodiments, this is realized by a maximum straight line distance between two points on the exterior surface and at the second end 22 of the introducer 2 being greater than 1 cm, more preferably greater than 1.5 cm, more preferably greater than 2 cm, more preferably greater than 2.5 cm, more preferably between 2.5 cm and 4 cm, more preferably between 3 cm and 3.5 cm, and most preferably is about 3.4 cm.

According to one embodiment, the wall of the introducer 2 may have a thickness of approximately 1 mm at the second end 22 of the introducer 2. However, greater thicknesses are also possible.

Further, the passageway 23 of the introducer 2 may have the same or a similar size at the second end 22 to prior art devices but the surface area may be otherwise increased. For example, the wall of the introducer 2 at the second end 22 may have an increased thickness. Additionally or alternatively, a flange may extend from the wall defining the opening, preferably extending from the exterior thereof, more preferably, substantially perpendicular to the passageway 23 so as to sit generally flush or parallel with the wall of the uterus forming the fundus. Additionally or alternatively, the introducer 2 may comprise a softened or more compressible portion at the second end 22. For example, an overmolding of softer material may be provided about the rim formed about the opening at the second end 22. Additionally or alternatively, the rim may be rounded to remove sharp edges.

As shown, at least a portion of the exterior surface of the introducer 2 may be cylindrical. For the avoidance of doubt, the at least a portion of the exterior surface that is cylindrical may be the entire or substantially the entire exterior surface. The cylindrical portion may extend from the first end 21 to a point distal from the first end 21 and between the first and second ends 21, 22, or may extend from the second end 22 to a point distal from the second end 22 and between the first and second ends 21, 22. Further still, at least a portion of the exterior surface may form a cylinder that is located between the first and second ends 21, 22 but is spaced from the first and second ends 21, 22, the latter being shown in the illustrated embodiments.

The cylindrical portion may have a constant radius. This radius may be, for example, between 1 cm and 1.5 cm, preferably about 1 cm.

While generally circular profiles are preferable for the introducer 22 so as to be received by the patient without undue discomfort, the disclosure is not limited thereto. Further, the cross-section of the introducer 2 may vary along at least part of its length between the first and second ends 21, 22. For example, the introducer 22 may be somewhat flattened so as to have a generally oval cross-section, at least for the exterior surface.

According to a preferred embodiment as illustrated in FIGS. 14-18, the exterior surface of the introducer 2 is generally bulbous at the second end 22. Providing a bulbous or similarly contoured surface at the second end 22 of the introducer 2 helps to remove sharp edges that can cause injury and further serves to increase the surface area of the end wall at the second end 22 of the introducer 2. This means that a greater force would generally be required to cause injury to a patient. Non-bulbous arrangements are also possible but bulbous configurations are preferred as they assist in insertion and removal. For example, other generally rounded, flared configurations may be used such as circular or elliptical cones, truncated at the base to join the narrower cross-section part of the introducer 2. Generally conical shapes that transition from being elliptical at the second end 22 to circular or more circular at the truncated base that joins to the remainder of the introducer 2 are also possible. Non-round shapes but with rounded corners may be used. These still provide for less concentration of forces and hence likelihood of injury than prior art devices but will tend to not do so to quite the same extent as more generally rounded shapes.

To compare the forces required to cause perforation between the embodiment of FIGS. 14-18 and equipment used in prior art methods, testing was performed. A ballistics gel uterine model was created using ballistics gel with thickening agents. This consisted of 120 grams of gelatine powder dissolved in 500 mL of hot water. 15 mL of sodium metabisulfite was added as a preservative. Sixty mL of granular psyllium was added as a thickening agent. An additional 500 mL of cool water was added once the gelatine powder had dissolved. A portion of the mixture was poured into a 1 litre plastic container. Seven folded paper towels were then inlayed on top of the mixture, and the remainder of the mixture poured on top. The gel was then left undisturbed to solidify. Once the gel had solidified, testing commenced.

The models were tested to a known calibration standard for 20-54 N of force required for the insertion device for a copper IUD to perforate the uterus. (Goldstuck and Wildemeersch. Role of uterine forces in intrauterine device embedment, perforation, and expulsion. International Journal of Women's Health 2014:6 735-744.) The gelatin models were calibrated to this standard using a Choice TT380 IUD and accompanying inserter. Perforation forces were measured using a Wedderburn WSDFG digital force gauge.

Twenty insertions were tested in this manner of both the prototype PPIUD device (per FIGS. 14-18) and using the standard PPIUD insertion method using forceps (i.e. forceps while holding an IUD) as described by Goldthwaite et al (Goldthwaite L M, Sheeder J, Teal S B, Tocce K M. Comfort With Skills and Knowledge After Immediate Postpartum Intrauterine Device Training. Obstet Gynecol. 2016 October; 128 Suppl 1:6S-11S.). Perforation force measurement medians were compared using the Mann-Whitney test.

The forces required to cause perforation or puncturing of the surface of the gel were noted and the results are presented in Table 1 below.

TABLE 1
Perforation force comparison (in N)

| | Model 1 | Model 2 | Model 3 | Model 4 | Model 5 | Model 6 | Model 7 | Model 8 | Model 9 | Model 10 |
|---|---|---|---|---|---|---|---|---|---|---|
| Calibration | 46.6 | 38.2 | 26.8 | 22.9 | 18.1 | 25.9 | 20.2 | 20.9 | 20.5 | 24 |
| test | | | | | | | | | | |
| Sponge | 85.6 | 77.9 | 37.9 | 29.9 | 34.2 | 52.4 | 47.8 | 38.1 | 46.4 | 42.7 |
| forceps 1 | | | | | | | | | | |
| Sponge | 78 | 132.6 | 33 | 30.1 | 36.7 | 51.5 | 47.6 | 59.8 | 61.4 | 56.4 |
| forceps 2 | | | | | | | | | | |
| PP19 1 | 370.7 | 319.3 | 314.9 | 448.6 | 318.9 | 322.1 | 304.4 | 388.1 | 340.2 | 252.9 |
| PP19 2 | 404 | 394.9 | 352.5 | 379 | 329.6 | 354.6 | 343.9 | 431.9 | 410.7 | 346.4 |

Table 2 below shows the Mann-Whitney analysis.

TABLE 2
Mann-Whitney analysis

| Parameter: | Forceps | PP-19 |
|---|---|---|
| Mean: | 54.000 | 356.38 |
| # of points: | 20 | 20 |
| Std deviation: | 24.589 | 47.556 |
| Std error: | 5.498 | 10.634 |
| Minimum: | 29.900 | 252.90 |
| Maximum: | 132.60 | 448.60 |
| Median: | 47.700 | 349.45 |
| Lower 95% CI | 42.492 | 334.12 |
| Upper 95% CI: | 65.508 | 378.64 |

As can be seen, the perforation forces were much higher for PP19 1 and 2 samples (i.e. insertion device prototypes made in accordance with FIGS. 14-18) than for the forceps samples 1 and 2. The mean force for the forceps samples was 54.0 N and for the PP19 samples, 356.38 N. The medians were 47.7 N and 349.45 N, respectively. The test indicated that the force required to perforate the uterus using an embodiment per FIGS. 14-18 was about 7 times higher than using equipment used in prior art methods. Further, forces of the order of 350 N are considerable and would require considerable exertion by the medical professional inserting the IUD to cause perforation or puncturing, significantly limiting the likelihood of perforation in practice.

According to the embodiment as shown in FIGS. 14-18, the bulbous portion is formed by a generally sinusoidal contouring of the exterior surface when viewed in a longitudinal cross-section, such that the surface curves away or outward from a smaller dimensioned portion to increase the width of the introducer 2 moving towards the second end 22. According to preferred embodiments the surface transitions to a point of inflexion such that the width decreases slightly in a region from proximate the inflexion moving to the second end 22. The inflexion point may be about 1 cm from the second end 22 although it may be otherwise positioned or omitted.

The introducer 2 may comprise a grip portion 25 integrally formed therewith or coupled thereto, preferably at or proximate to the first end 21. The grip portion 25 may be formed by contouring of the outer surface of the introducer 2 at or proximate to the first end 21. The grip portion 25 may be used to assist generally in gripping the introducer 2 but especially during pushing of the pusher 3 or when pulling the introducer 2 out of the patient. Other configurations of the grip portion 25 are possible.

The introducer 2 may be configured such that at least a portion of the elongate passageway 23 is circular. The circular portion may extend from a non-round portion that extends from the first end 21 and the circular portion may extend to the second end 22. At least a substantial part of the passageway 23 may have a constant or substantially constant diameter, preferably of approximately 1 cm.

While continuing to be circular, the size of the passageway 23 may be increased at and proximate the second end 22. As shown, for example, in FIG. 15, the inner wall of the introducer 2 may define a concavity at the second end 22. The passageway 23 as shown is formed such that when the pusher 3 is partially inserted to a first predetermined extent, which may be established by an indicator 31 provided on the pusher 3 (see FIG. 18) reaching a predetermined point relative to the introducer 2 (e.g. the indicator 31 aligns with the first end 21 of the introducer 2), the arms of the IUD are retained in the narrower part of the passageway 23. In this position, the IUD may still be fully retained inside the narrower part of the passageway 23 or may project into the flared part of the introducer 2. The engaging features provided to the surface of the elongate passageway 23 are preferably provided to assist in retaining the IUD in this position. This can also be configured to provide haptic feedback to a user that the IUD is in the correct position as the arms of the IUD engage the engaging features. This is the configuration pre-insertion into the uterus. After insertion of the insertion device 1 into the uterus such that the second end 22 abuts the fundus, the pusher 3 is pushed further, to full insertion. A mechanical stop, as defined by the projections 32 on the pusher 3 in the illustrated embodiment, although other realisations will be apparent, the arms of the IUD are freed from the narrower part of the passageway 23. Due to the sloping walls of the concavity at the second end 22 of the introducer 2, the springing of the arms of the IUD to their rest position urges the IUD towards the fundus, aiding in properly locating the IUD.

Further, the additional space formed by the concavity means that if for some reason there is a problem with insertion of the IUD (e.g. the orientation of the IUD is incorrect), the IUD falls back into the space provided by the concavity, rather than being urged towards a wall of the uterus with potential injury to the patient (e.g. perforation) resulting therefrom. Consequently, the concavity is preferably dimensioned such that the IUD is able to be completely or largely contained within the concavity when the pusher 3 is fully inserted in to the introducer 2. Thus the passageway 23 may have a diameter of about 2 cm to 2.5 cm at the second end, preferably about 2.3 cm, and the region of increased cross-sectional area of the passageway 23 at and proximate the second end 22 may extend between about 2.5 cm and 3.5 cm, preferably about 3 cm. This is aided by the pusher 3 only loosely engaging the IUD i.e. the pusher 3 does not actually maintain an orientation of the IUD, other than preferably resisting the stem of the IUD becoming trapped between the pusher 3 and the passageway 23 of the introducer 2.

As will be appreciated, this means that the IUD is sheathed until it reaches its ultimate destination, reducing the likelihood of infection. Preloading of an IUD into the passageway 23 can further reduce risks of infection by avoiding direct handling of the IUD at the site it is inserted.

Preferably, when fully inserted, the pusher 3 does not extend beyond the second end 22 of the introducer 2. More preferably, it may be positioned inside the passageway 23 at a point remote from the second end 22. For example, the tip of the pusher 3 may be approximately 0.5 cm from the second end 22 of the introducer 2 when fully inserted. This avoids the tip of the pusher 3 contacting the uterus and potentially causing injury and this, along with the configuration of the concavity can help prevent the IUD being forcibly pushed against the uterus.

As shown for example in FIG. 18, the pusher 3 may comprise an elongate rod, preferably having a generally circular cross-section although other cross-sections are possible. The key requirements of the pusher 3 are that it is able to be received inside the passageway 23 and slide inwards and outwards thereof, urging an IUD along the passageway from the first end 21 to the second end 22 during use.

To prevent the string of the IUD from being captured between the pusher 3 and the walls defining the passageway 23, a sufficient gap is provided between the engaging walls, rather than there being a snug, piston-like engagement between the two. However, this gap is preferably not so large that the base of the trunk of the IUD can become trapped between said surfaces. According to one embodiment, this gap is about 1 mm or about 0.5 mm on each side of the pusher 3 when the pusher 3 is centrally positioned inside the passageway 23. This gap particularly ensures that the string is not caught between the pusher 3 and the walls of the passageway 23 as the pusher 3 is withdrawn, which could cause the IUD to be removed following insertion.

The pusher 3 may comprise a recess 33 in the end wall of the first end thereof i.e. the surface that pushes against the base of the trunk of the IUD. This recess 33 can serve to locate the IUD relative to the pusher 3, discouraging it from being urged into the gap between the pusher 3 and the walls forming the passageway 23. This can make urging of the IUD along the passageway using the pusher 3 easier and more reliable.

The first end of the pusher 3 may additionally or alternatively comprise a pair of notches 34 on opposing sides of the pusher 3. While non-essential, these can provide a preferred location for the strings of the IUD and also help to orientate the IUD relative to the pusher 3.

One or more indicia may additionally or alternatively be provided to the outer surface of the introducer 2. These may indicate or limit an extent to which the introducer 2 has been inserted into the patient.

According to a presently preferred embodiment, the introducer 2 has a length of approximately 28.5 cm and the pusher has a length of approximately 33 cm.

The introducer 2 and/or the pusher 3 may be formed from plastics. Alternatively, the introducer 2 and/or the pusher 3 may be formed from a metal. Suitable grade materials for use in such a medical procedure will be apparent to those skilled in the art. Metals may be preferable where the device 1 is configured for use as sterilising processes may have no or a lesser detrimental effect on metals.

Use of the insertion device has been described when describing features of the device but for completeness, may be used as follows.

An IUD, is inserted into the elongate passageway 23 of the introducer 2 of the insertion device 1 at the first end 21 thereof with the end of the trunk of the IUD from which the arms extend being inserted first. The pusher 3 is then urged against the base of the trunk of the IUD until the pusher 3 is partly received in the elongate passageway 23 at the first end 21 of the introducer 2. The pusher 23 is then urged further along the elongate passageway 23 such that the arms are still retained in the narrower part of the passageway 23. As disclosed previously, the insertion device 1 may be sold in this configuration with the pusher 3 inserted or outside of the passageway 23. The insertion device 1, along with the IUD, is then inserted into the uterus such that the second end 22 of the introducer 2 abuts the fundus. The pusher 3 is then further urged into the elongate passageway 23 such that the IUD springs free of the introducer 2. The pusher 3 is then retracted from the introducer 2 and finally the introducer 2 is removed.

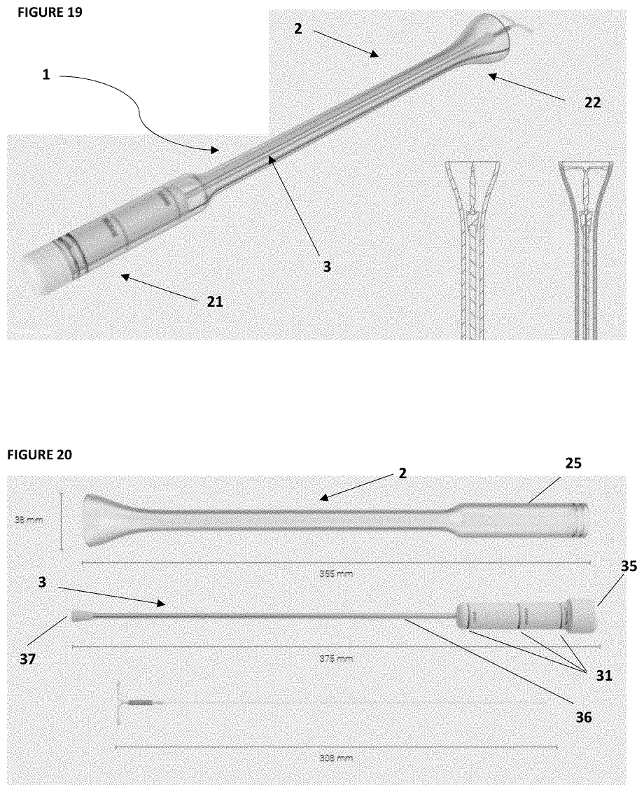

FIGS. 19-26 show an alternative embodiment. In this and later embodiments, the same reference numerals are used as for FIGS. 14-18, where possible.

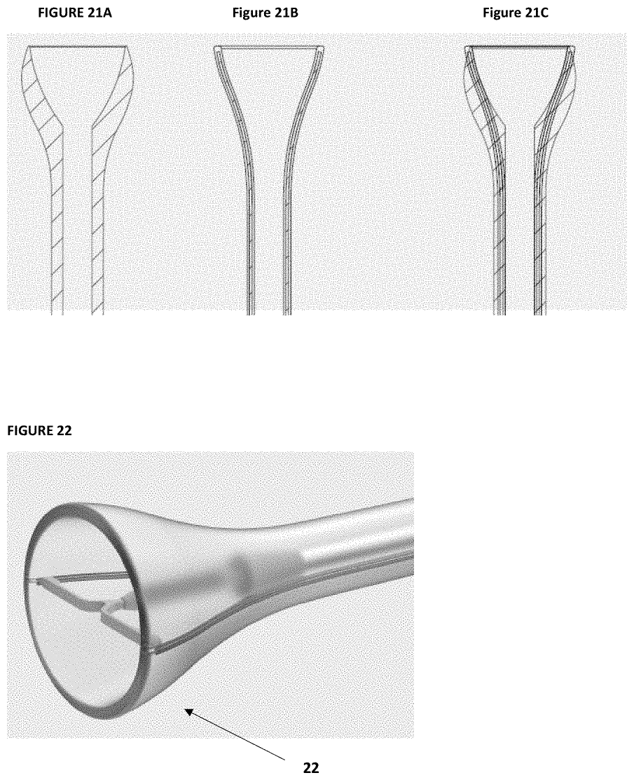

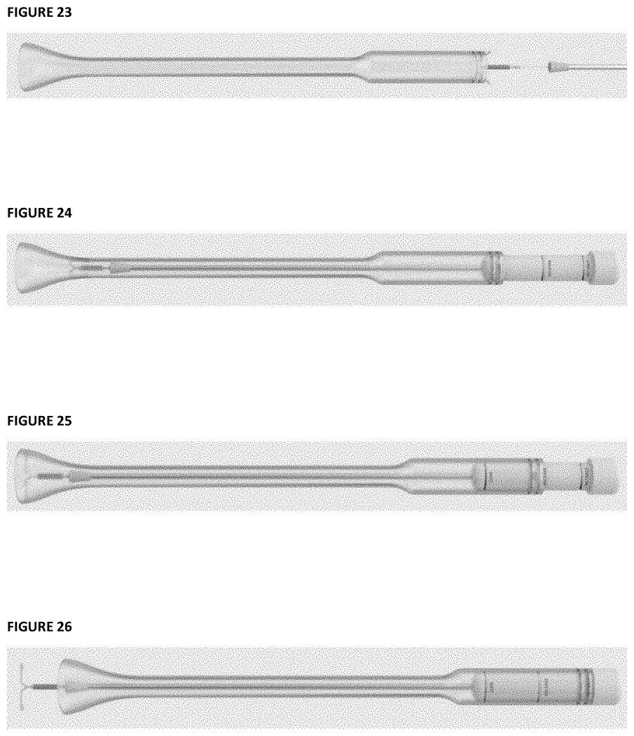

FIG. 19 is a perspective view of an alternative IUD insertion device 1 including a modified introducer 2 and pusher 3. FIG. 19 includes inset orthogonal sectional views of the second end 22 of the insertion device for reference. FIG. 20 is a plan view of the components shown in FIG. 20 separated from each other and with example dimensions provided. FIGS. 21A-21C are cross-sections of the second ends 22 of the introducer of FIGS. 14-18 (FIG. 21A) positioned side-by-side a similar view of the embodiment of FIGS. 19-26 (FIG. 21B), with these two views then superimposed for comparison purposes (FIG. 21C). FIG. 22 is an enlarged perspective view of the second end 22 of the introducer 2. FIGS. 23-26 show positions of the various elements during use of the insertion device 1 in inserting an IUD inside a patient.

As is apparent from the Figures, the general structure is the same between these two embodiments and only differences will be discussed.

In the embodiment of FIGS. 19-26, a different grip portion 25 is provided, in the form of a cylindrical body extending from the first end 21 of the introducer 2. As shown, this tapers to a smaller diameter in the main, central part of the introducer 2 before flaring outwards near the second end of the introducer 2.

In the embodiment of FIGS. 19-26, the wall of the introducer 2 has a substantially constant thickness (preferably of about 3 mm) rather than the more significant changes per the embodiment of FIGS. 14-18. This is shown, for example, in FIGS. 21A-21C for the second end of the introducer 2, but is also apparent from FIGS. 19, 20 and 22-26 since the introducer 2 is transparent or substantially transparent in these Figures. Transparency of the introducer 2 is optional but aids in use of the device by clearly showing the position of the pusher 3 and the IUD inside the introducer 2.

Since the wall of the introducer 2 of FIGS. 19-26 has a substantially constant thickness, it will be apparent that the grip portion 25 defines a larger diameter section of the passageway 23 that tapers to a smaller diameter before flaring out again at the second end 22.

FIGS. 21A-21C compare the sections of the second ends of the introducers 2 of the first and second illustrated embodiments. The external surface is less bulbous in the embodiment of FIGS. 19-26 and internally, the transition from the flared, larger diameter portion to the small diameter portion is smoother. This alternative design provides easier manufacturing by injection moulding but can also help prevent the string of the IUD getting caught between the pusher 3 and the surface of the introducer 2 defining the passageway 23 which can result in the IUD being pulled out of position after insertion.

Referring to FIG. 19, the introducer 2 is shown as including a line 25. This is also shown in FIG. 22 but from this Figure, it is apparent this feature is provided on both, opposing sides of the introducer 2. According to preferred embodiments this feature comprises a pair of longitudinal grooves that oppose each other inside the introducer 2 and that serve to receive and orientate projections of the IUD inside the introducer 2, better ensuring correct orientation when inserted against the fundus. The introducer 2 may be formed from two parts that are joined at this point, for example, by ultrasonic welding. As with the first embodiment, as an alternative to the grooves, the passageway 23 may otherwise be non-round or provide for orientation of the IUD inside the passageway 23, such as by having an oval cross-section.

As shown, for example, in FIG. 20, the pusher 3 comprises a handle part 35, rod 36 and IUD engaging tip 37. Markings 31 are provided on a part of the body that forms the handle part 35 but which are received inside the introducer 2. According to this embodiment, the rod 36 is formed from a metal (e.g. aluminium or a surgical grade steel) which enables the pusher 3 in this region to have a smaller diameter but retain its stiffness. This smaller diameter can also help prevent the string of the IUD catching between the pusher 3 and the introducer 2. The tip 37 of the pusher 3 has a larger diameter to prevent the IUD becoming positioning between the pusher 3 and the inner surface of the passageway 23 of the introducer 2. Similar to the first illustrated embodiment, the tip 23 may comprises a recess to locate the IUD and/or notches to receive the strings of the IUD.

While this second embodiment has generally been described with the components having circular sections, the invention is not so limited. Again, at least oval and rounded profiles are within the scope of the invention with the only real limitations being to avoided sharp edges that may cause injury to a patient and to avoid catch or sticking points for the IUD or the string.

FIGS. 23-26 sequentially show how the parts move during the insertion process. FIG. 23 shows initial insertion of an IUD in the first end 21 of the introducer 2. The pusher 3 is then inserted into the first end of the introducer 2 such that it urges the IUD towards the end, but still inside, the narrow part of the passageway 23 as shown in FIG. 24. The apparatus is preferably inserted into the patient in this position until the second end 22 of the introducer 2 is positioned against the fundus. In this position, the marking 31 farthest from the handle part 35 is aligned with the first end 21 of the introducer 2 or a marking or other feature on the introducer 2 that is proximate the first end 21.

With the apparatus in the correct position against the fundus, the pusher 3 is pushed farther into the introducer 2 such that the second (the middle one) of the markings 31 is aligned with the reference point on the introducer 2. During this movement, the IUD is released from the confines of the narrower part of the passageway 23 and is free to move within the flared space at the second end 22 of the introducer 2. Continued pressure to the handle part 35 urges the pusher 3 to its maximum extent inside the introducer 2 and pushes the IUD free of the passageway 23. Again, a mechanical stop is provided to avoid the pusher 3 being pushed in too far, this time in the form or a step in or adjacent the handle part 35 that comes into abutment with the first end 21 of the introducer 2 when fully inserted.

As will be appreciated, features may be blended or swapped between the different embodiments. For example the pusher of the first embodiment may be exchanged with that of the second embodiment and vice versa, with appropriate modifications as required.

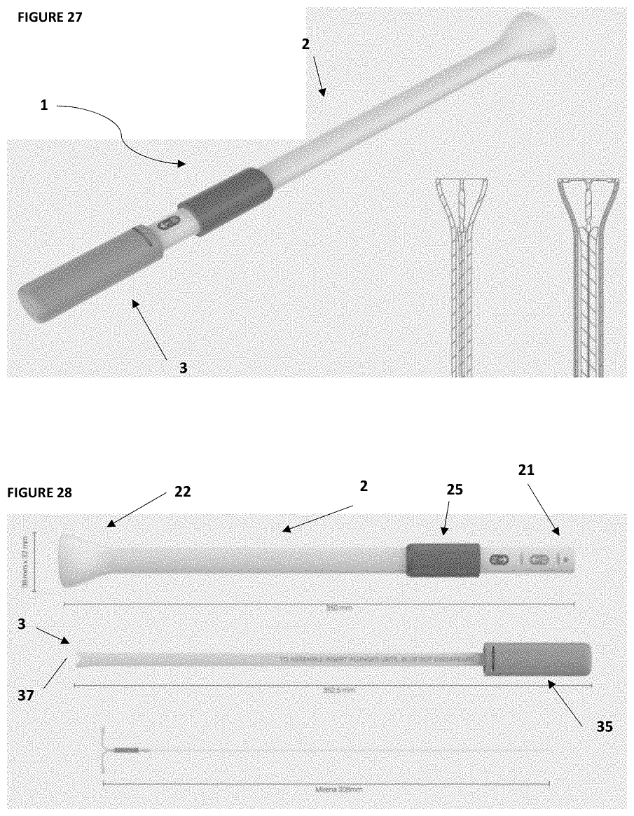

FIGS. 27-34 illustrate a third embodiment of an IUD insertion device 1. Again, only points of difference with the previous embodiments will be discussed and reference is again made to the earlier description.

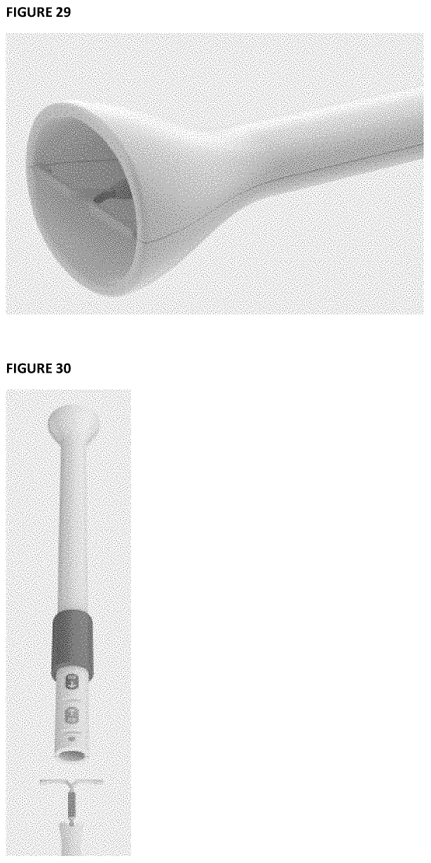

FIGS. 27 and 28 are perspective and plan views of the apparatus, the latter showing some preferred dimensions. Similar to FIG. 19, FIG. 27 includes inset orthogonal sectional views of the second end 22 of the insertion device for reference. FIG. 29 shows an enlarged view of the second end 22 of the introducer 2 with an IUD immediately adjacent the second end 22. FIGS. 30-34 are sequential diagrams showing use of the apparatus for IUD insertion.

As best seen in FIGS. 27 and 29, the section or profile of the introducer 2 of this embodiment is different. Namely, it is flattened such that at least a substantial part of the introducer 2 between the first and second ends 21, 22 has upper and lower surfaces that are closer together than the side surfaces, with the upper and lower surfaces being flatter than the more rounded side surfaces. Similar to the second illustrated embodiment, at least a substantial portion of the wall forming the introducer 2 has a substantially constant thickness and so contouring of the external surface of the introducer is reflective of the configuration of the passageway 23 inside the introducer 2.

As shown in FIG. 28, the introducer 2 includes a grip portion 25 that is spaced apart from the first end 21 of the introducer 2. The portion of the introducer 2 in this embodiment between the first end 21 and the grip portion 25 is received inside a handle portion 35 of the pusher 3, as shown in FIGS. 32-34. In the Figures, this pusher 3 is shown as comprising a cylindrical plastic rod having a dimension much smaller than that defined by the passageway 23 such that there is space between the rod and the passageway 23 in use to prevent capture of the string of the IUD. Again, the tip 37 of the pusher 3 may comprise the previously disclosed recess 33 and, or notches 34.

Again, as shown best in FIG. 29, the inner surface of the introducer may comprise longitudinal grooves that serve to orientate the IUD inside the passageway 23. Again, the introducer 2, may be formed in two parts as shown by this line extending on both the inside and outside surfaces of the introducer 2.

The flared second end 22 of introducer may have the same profile or cross-section as the remainder (or at least a substantial portion of the introducer between the two ends 21, 22) but enlarged. Alternatively, the introducer 2 may transition to a circular (more particularly annular) or more rounded or circular section in this region of the introducer 2.

FIG. 30 shows the introducer 2 with an IUD about to be loaded in the passageway 23. The non-rounded cross-section of the passageway 23 provides the user with an intuitive pointer as to the proper orientation of the IUD and the profiling of the passageway 23 serves to correct improper alignment.

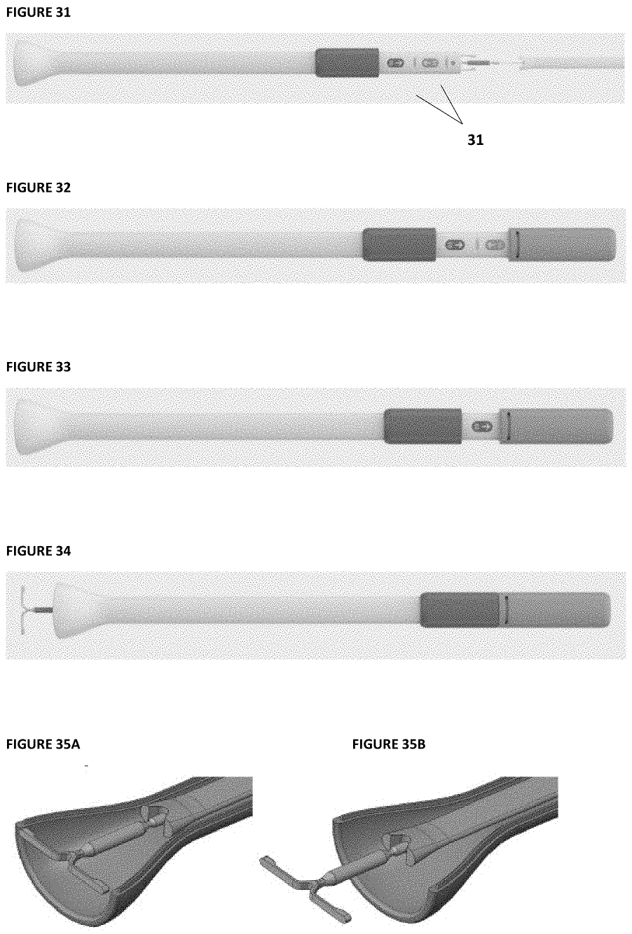

FIG. 31 shows the IUD slightly after insertion in the first end 21 of the introducer 2 with the pusher 3 beginning to urge the IUD along the passageway 23 inside the introducer 2. FIG. 32 shows the apparatus in the “loaded position”, similar to that of FIG. 24, with the IUD still captured within the confines of the narrower part of the passageway 23 and the patient facing end of the handle portion 35 aligned with the marking 31 on the introducer 2 nearest the first end 21. The device is now ready for insertion inside a patient.

With the second end 22 of the introducer 2 abutting the fundus, the pusher 3 is pushed to the next marking 31, as shown in FIG. 33, causing the IUD to be pushed out from the narrower part of the passageway 23 to the enlarged, flared part. Continued pushing of the pusher 3 brings an end of the handle portion 35 into abutment with the grip portion of the introducer 2, acting as a mechanical stop to prevent continued insertion of the pusher 3, and potential injury to the patient, as shown in FIG. 34. In this movement, the IUD is urged out of the flared portion at the second end 22 of the introducer 2.

Again, features of this embodiment may be transposed to other embodiments and vice versa. For example, the configuration of the pusher 3 may be swapped between embodiments, and/or the configuration of the mechanical stop used to limit movement of the pusher 3 towards the patient.

FIGS. 35A and 35B show the patient engaging ends of the pusher 3 and introducer 3 according to one embodiment with an IUD shown in position and the upper half of the introducer 2 removed to show internal detail. Features shown here may be incorporated in any of the embodiments shown or described herein.

As shown in FIGS. 35A and 35B, the tip of the pusher 3 is U-shaped so as to receive and locate the base of the IUD and provide a path for the strings. The pusher 3 is shown as having a non-circular cross-section, rather it is rounded but flattened so as to be generally oval. Alternatively, as with previous embodiment, the main shaft part of the pusher may be replaced with a circular rod, such as of metal. The pusher 3 is shown as having an increased cross-sectional area near the IUD engaging end thereof to better ensure proper contact with the base of the IUD and prevent the base of the IUD being captured between the pusher 3 and the surface defining the passageway inside the introducer 2. The upward facing edges of the introducer 2 part are shown as including tongues. These may engage with complementary tongues to facilitate joining of the parts although other contructions are possible.

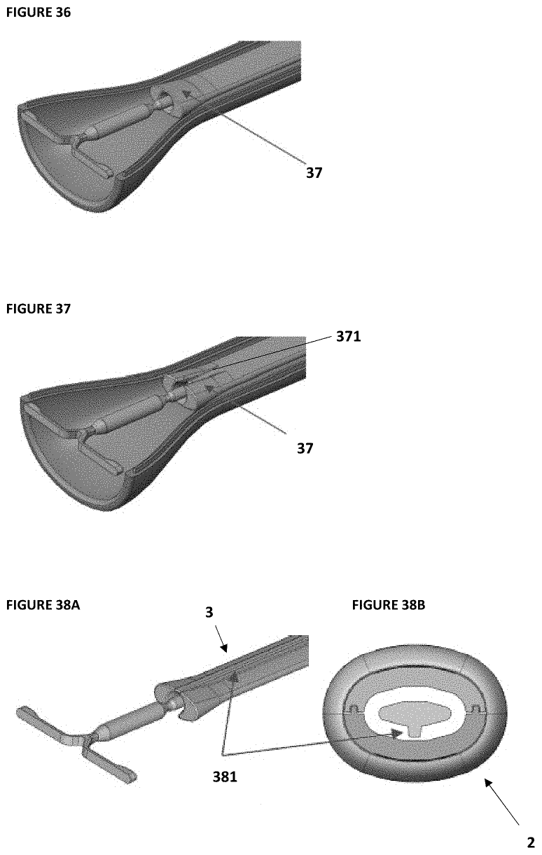

FIG. 36 is a view similar to that of FIG. 35A but showing an alternative embodiment of the IUD engaging end of the pusher 3. Rather than the U-shaped profile of FIGS. 35A and 35B, the end of the pusher 3 has a depression that forms a bowl i.e. it completely encircles the base of the IUD. This better locks the base of the IUD in position relative to the end of the pusher 3, preventing the IUD becoming trapped between the engaging walls of the pusher 3 and introducer 2.

FIG. 37 is a modified form of the FIG. 36 embodiment. According to this embodiment, a groove 371 is added to receive the string of the IUD. This Figure better shows how the cross-section of the pusher 3 varies approaching the IUD engaging end thereof.

FIGS. 38A and 38B show a modified form of the FIG. 37 embodiment. In this embodiment, a reinforcing rib 381 is provided to the pusher 3, preferably to the underside thereof and/or the side opposite to that which includes the groove 371. This provides stiffening or reinforcement for the pusher 3.

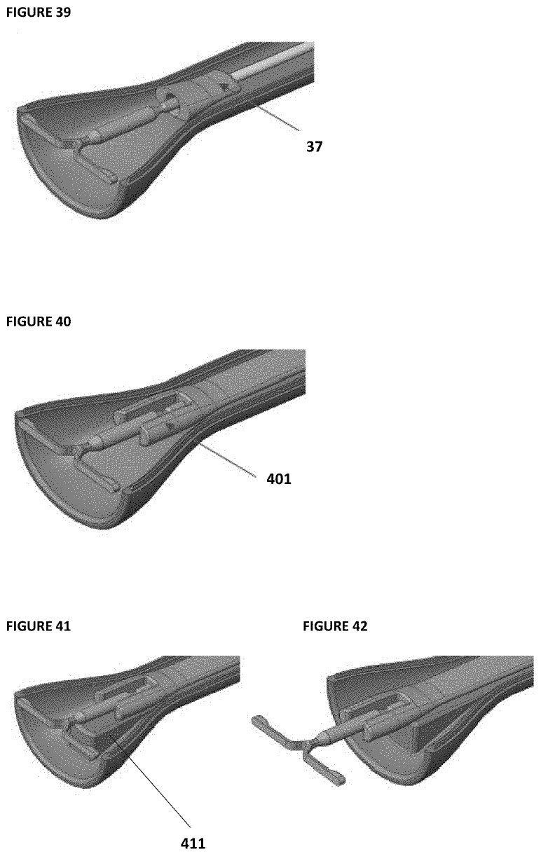

In the embodiment of FIG. 39, the main shaft portion between the two ends of the pusher 3 is replaced with a metal rod e.g. of medical grade stainless steel. This provides stiffness, removing tolerance issues that may be present when the pusher 3 more completely fills the passageway 23. The IUD engaging end of the pusher 3 is configured per the embodiment of FIG. 36 but this replacement of the main shaft of the pusher 3 with a metal rod may be applied to the other embodiments also.

FIG. 40 shows another alternative configuration of the IUD engaging end of the pusher 3. In this embodiment, finger-like extensions or prongs 401 are configured to receive the stem or trunk of an IUD therein. As shown in FIG. 40, these fingers preferably extend along a substantial portion of the length of the IUD stem (e.g. about half of the length of the stem). This embodiment provides for easier initial insertion of the IUD and pusher 3 into the introducer 2 with the desired relative poisoning by allowing a simple forefinger and thumb grip of both the IUD engaging end of the pusher 3 and the IUD at the same time, as the two are inserted into the introducer 2. The groove or channel 371 may be added to this embodiment to provide a location for the string of the IUD. Further, a blending of this embodiment with that of FIG. 36 or 39 would be possible, whereby the prongs 401 extend from the opening of the bowl.

A further embodiment combines features of the embodiment of FIG. 40 with the embodiment of FIGS. 38A and 38B. More particularly, the pusher 3 may optionally comprise a longitudinal projection along the underside thereof that is received in and slides along a complementary recess or groove in the lower surface of the passageway 23. Additionally or alternatively, a portion of the upper surface of the pusher 3, preferably extending from or proximate from the IUD engaging part of the pusher 3, is recessed so as resemble a tray. Additionally or alternatively, the lateral upper edges of the pusher 3 may include walls that extend up from the main body of the pusher 3 so as to extend farther up the sides of the passageway 3. Additionally or alternatively, the outer sides of at least a portion of the pusher 3 that may be received in complementary slots in the side walls of the passageway 23. These features may provide for smoother movement of the pusher 3 inside the passageway 23 and/or assist in preventing the string of the IUD becoming trapped or caught between the pusher 3 and the passageway 23. Further, it can provide for greater control of the orientation of the pusher 3 and consequentially also the IUD inside the introducer 2.

Note that here and elsewhere in this specification, reference to one component having a particular engaging or mating feature that engages or mates with a complementary or corresponding feature of another component is to be taken as also being disclosure of the reverse formation of the mating. For example, if a first component is said to have a groove that engages a rib of a second component, this is to be taken as also disclosing a rib on the first component that engages a groove on the second component.

FIGS. 41-45 provide different views of a further alternative embodiment. The additional features of this embodiment are illustrated in relation to the FIG. 40 embodiment but it will be appreciated that these features may also be added to the other embodiments disclosed herein.

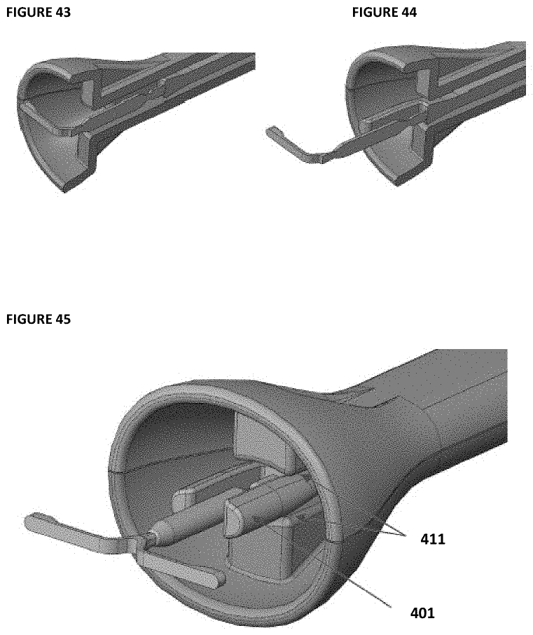

FIGS. 41 and 42 are perspective views with the upper half of the introducer 2 removed to show internal detail and with the pusher 3 in intermediate and extended positions, respectively. FIGS. 43 and 44 are corresponding views to those of FIGS. 41 and 42, respectively, but with the right half of the apparatus cut away to show internal detail, instead of the top part of the introducer 2. FIG. 45 is a perspective view of the assembled apparatus with the pusher 3 shown in the extended position, similar to FIGS. 42 and 44.

The embodiment of FIGS. 41-45 includes a projection 411 on each of the opposing upper and lower surfaces of the passageway 23 in the flared or bulbous portion of the introducer 2. As shown, the projections 411 preferably extend from the end of this portion nearest the first end 21 of the introducer 2 part way towards the second end 22 of the introducer 2. According to the illustrated example, the projections may extend approximately halfway along the flared or bulbous portion of the introducer 2. While not limited thereto, the projections 411 may effectively extend the narrower portion of the passageway 23, as shown in FIGS. 43 and 44 but only above and below the IUD engaging end of the pusher 3. Further, as best shown in FIG. 45, the opposing faces of the projections may be concave, in the form of a longitudinal depression, serving to centre the pusher 3 in this portion of the passageway 23. This embodiment may provide for greater control of the position of the IUD during insertion but provides large space either side of the projections for the strings of the IUD to pass.

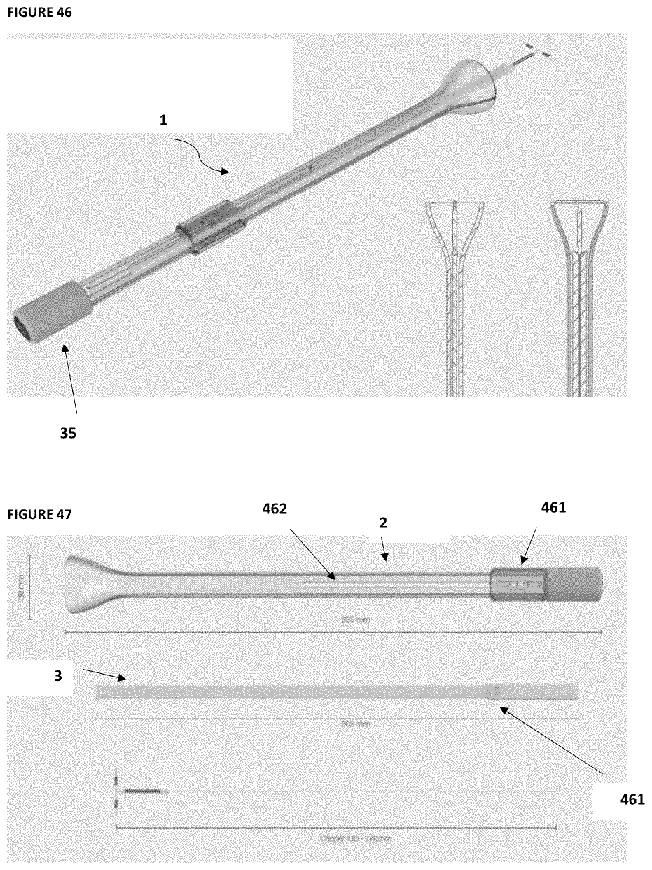

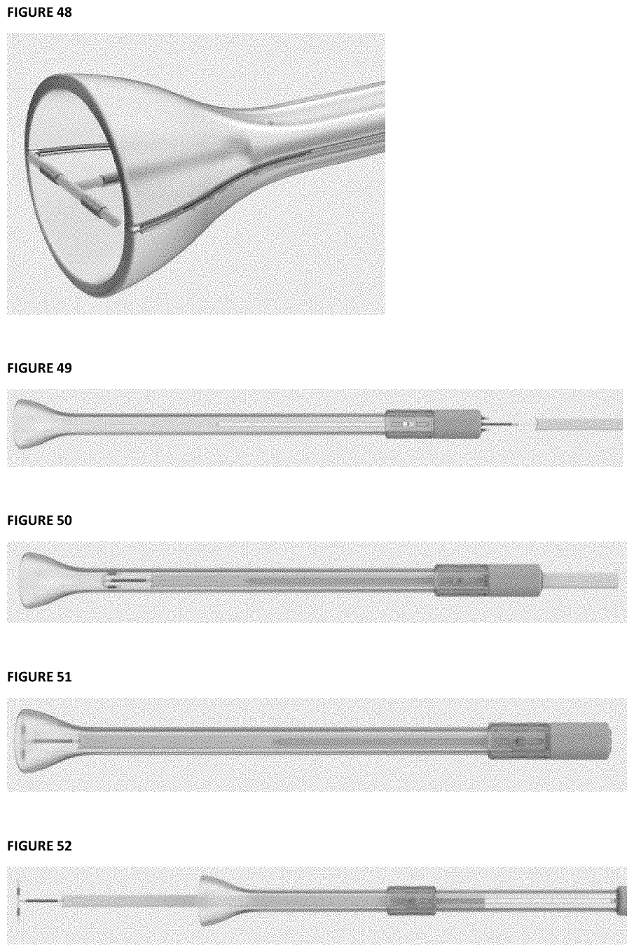

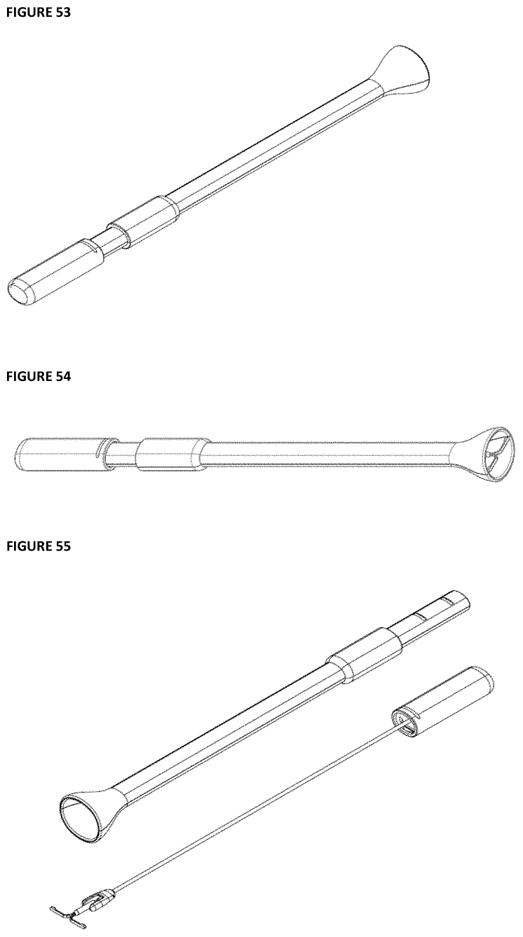

FIGS. 46 to 52 provide various views of another embodiment. FIG. 46 is a perspective view in the extended position including inset orthogonal sectional views of the second end 22 of the insertion device for reference similar to FIG. 19, FIG. 47 is a plan view of the components disassembled and showing example dimensions. FIG. 48 is an enlarged perspective view of the patient engaging end of the apparatus. FIGS. 49-52 show the apparatus in different positions as would occur in use.

This embodiment differs somewhat from the other embodiments, namely in the way in which the pusher 3 is extracted after insertion of the IUD.

More particularly, introducer 2 includes a slider 461. The slider has one or more projections that are slidably received in one or more corresponding longitudinal slots 462 through the wall of the introducer 2. These allow the slider to move forwards and backwards, bounded near the first end 21 by the handle portion 35 and between the first and second ends 21, 22 by the slot(s) 462 terminating.

The or at least one the projections of the slider 461 is further configured to engage a mating portion of the pusher 3 when the pusher 3 is inserted into the introducer 2. For example, as shown in FIG. 47, a surface of the pusher may comprise a depression 463 that mates with the or one of the projections that extend through the slot(s). Thus, when the parts are engaged, movement of the slider moves the rod relative to the introducer 2, albeit the relative movement between the introducer 2 and the pusher 3 is limited after engagement due to the limited movement of the slider 461. That said, the pusher 3 is preferably separable or disengageable from the introducer 2 for reuse, for example, by applying a greater pulling force.

With reference to FIGS. 49-52, the IUD is inserted in the first end 21 of the introducer 2 in the usual way, per FIG. 49. The pusher 3 is then urged into the introducer 2 until the IUD reaches the loaded position with the apparatus ready for insertion inside a patient. After the second end 22 of the introducer 2 is located against the fundus, the pusher 3 is fully inserted into the introducer 2 as shown in FIG. 51. In this position, the pusher 3 becomes engaged with slider 461.

The handle portion 35 is then pulled away from the slider 461 which is held in a fixed position, exposing the IUD for capture adjacent the fundus. Continued pulling of the handle portion 35 brings the slider 461 to the end of the slots such that the pusher 3 is then also extracted from the patient, but in a single action.

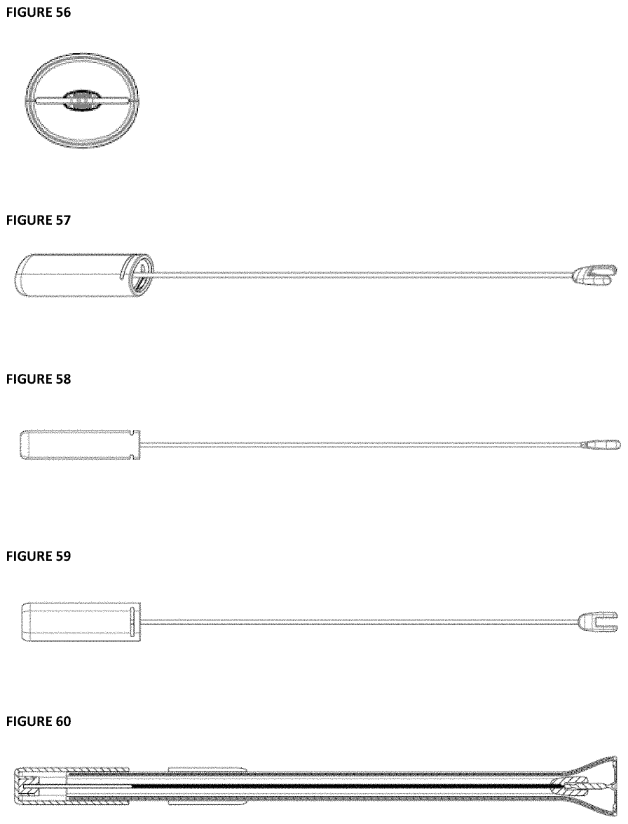

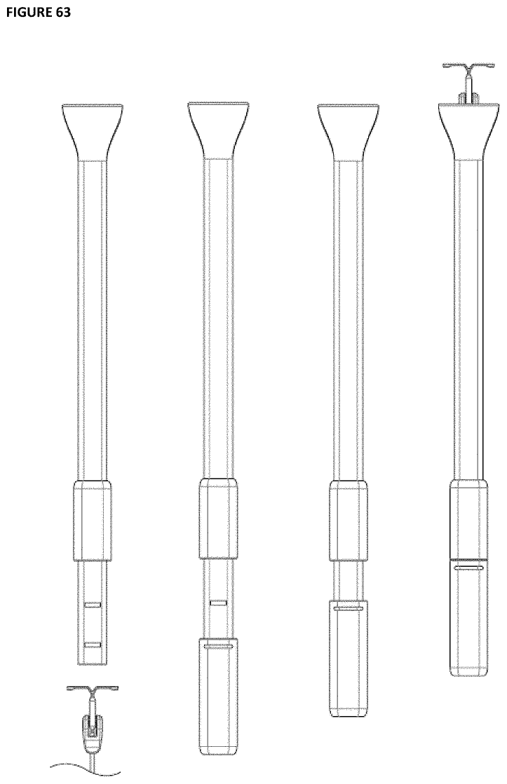

FIGS. 53 to 63 show an alternative embodiment of an IUD insertion device 1. Many of the features of this embodiment are common to earlier embodiments and only points of difference will be described. Further as with the other embodiments, features of this embodiment may be transposed to other ones of the embodiments and vice versa.

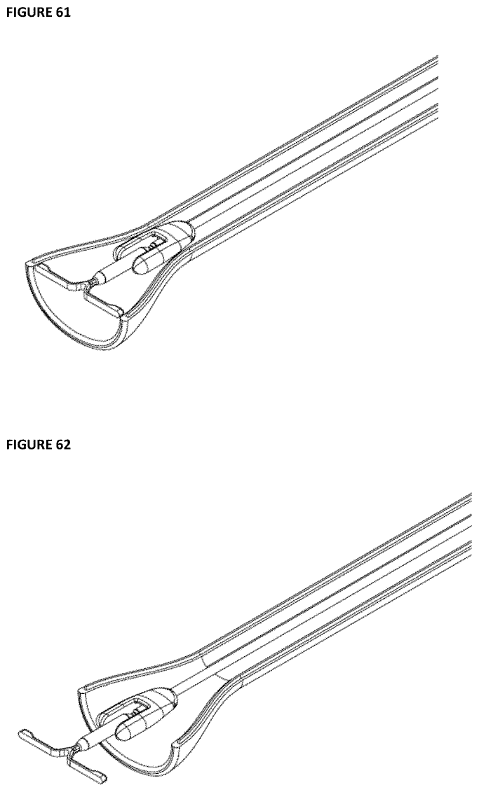

This embodiment is most similar to that of FIG. 27 but the pusher 3 includes a slim line rod, preferably of metal, to which a tip of the pusher 3 and the handle are attached. As best shown in FIG. 61, the tip of the pusher 3 includes the prongs as previously discussed but a depression is formed between the prongs (or a walls extend between the prongs) to create a recess in which the base of the IUD is received and restricted from translation perpendicular to the axis of the rod.

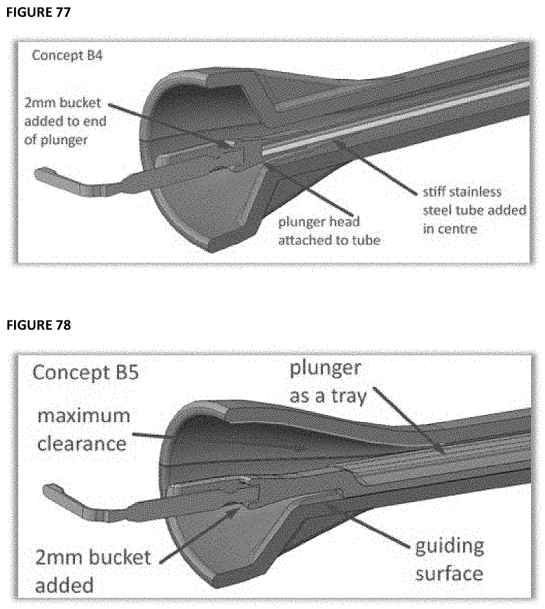

This may be better understood with reference to FIG. 77 which is a sectional view of essentially the same embodiment except with projections extending from the bottom and top of the inside of the flared portion to control a position of the tip of the pusher 3. This ma define a cavity of, for example, 2 mm for the base of the IUD to be received in.

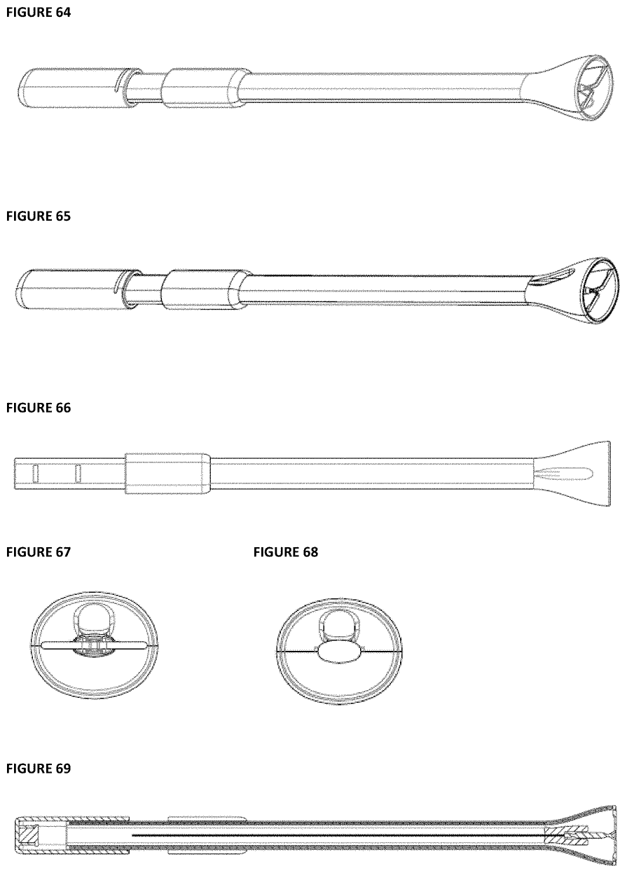

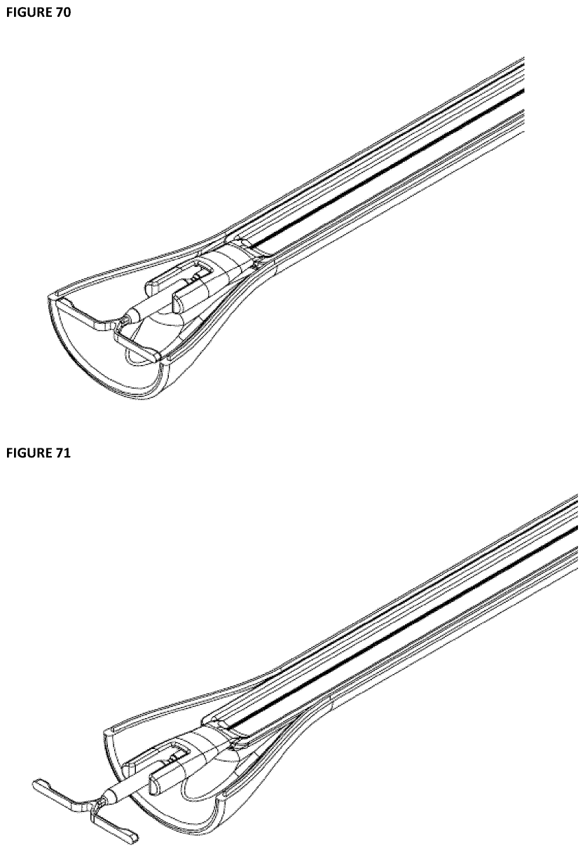

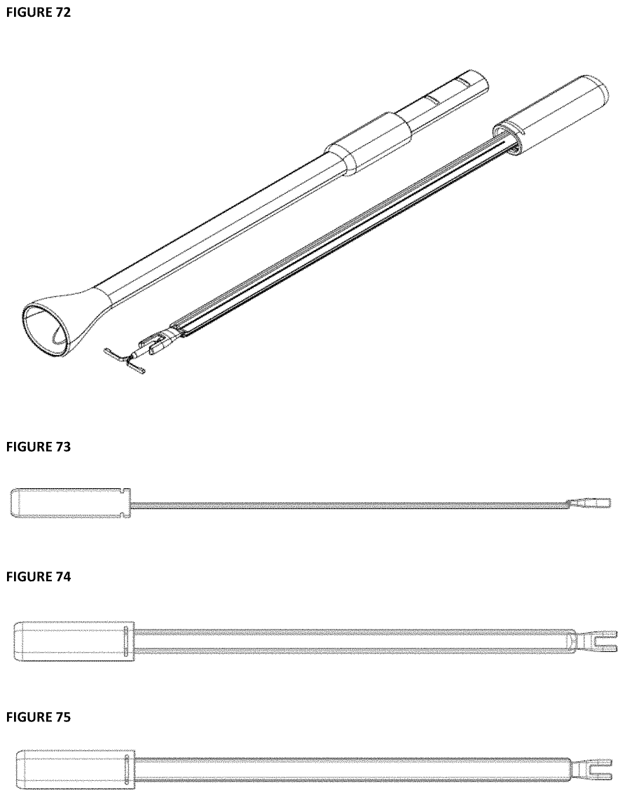

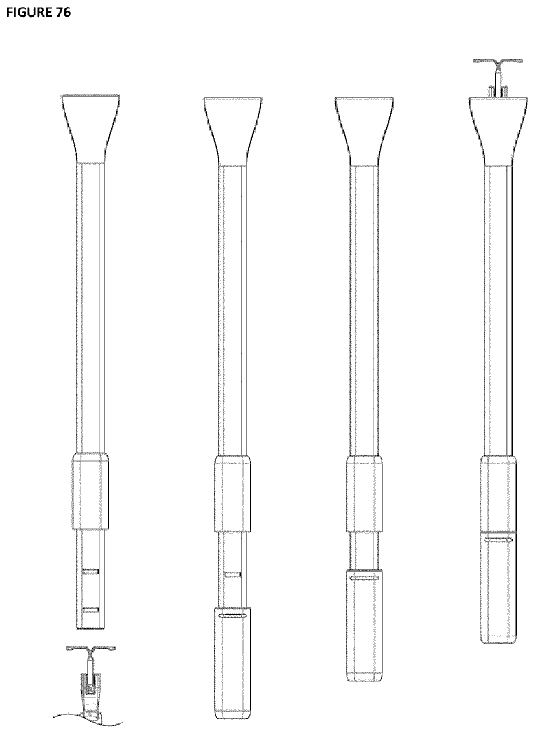

FIGS. 64 to 76 show an alternative embodiment of an IUD insertion device 1. Many of the features of this embodiment are common to earlier embodiments and only points of difference will be described. Further as with the other embodiments, features of this embodiment may be transposed to other ones of the embodiments and vice versa.

Notably in this embodiment, a depression is former in the lower, outer surface of the flared portion as shown in FIG. 65. This translates to a projection inside the flared portion of the introducer 2 as shown in FIGS. 67 and 68 (end views with and without an IUD present, respectively), and 71 and 72.

While relating to a slightly modified version of this embodiment, FIG. 78 provides further detail for the patient-engaging end of the IUD insertion device.

In the embodiment of FIGS. 64 to 76, the main part of the pusher between the tip and the handle is essentially in the form of a tray i.e. it is essentially planar but is preferably provided with raised edges on its upper surface and/or a ridge on its lower surface (or vice versa) so as to provide some rigidity to the element and/or to provide better sliding engagement with the introducer 2.

The “tray” configuration of the pusher along with the projection provided inside the flared portion of the introducer only being on the lower part thereof and not the upper and lower parts as shown in FIG. 77, reduces the possibility of the string of the IUD becoming caught between the pusher and the introducer which can lead to a failed or incorrect insertion of an IUD.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

Reference to any prior art in this specification is not, and should not be taken as, an acknowledgement or any form of suggestion that that prior art forms part of the common general knowledge in the field of endeavour in any country in the world.

It should be emphasized that many variations and modifications may be made to the embodiments described herein, the elements of which are to be understood as being among other acceptable examples. All such modifications and variations are intended to be included herein within the scope of this disclosure and protected by the following claims. Further, nothing in the foregoing disclosure is intended to imply that any particular component, characteristic or process step is necessary or essential.

While the methods and devices described herein may be susceptible to various modifications and alternative forms, specific examples thereof have been shown in the drawings and are herein described in detail. It should be understood, however, that the invention is not to be limited to the particular forms or methods disclosed, but, to the contrary, the invention is to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein.

1. An insertion device for an IUD, the insertion device comprising:
an introducer having an elongate hollow body and a first end and a second end, the hollow body defining an elongate passageway extending from the first end to the second end and defining openings at the first end and the second end; and
a pusher provided or configured to be provided inside the elongate passageway such that the pusher is movable through or along the passageway,
wherein the passageway is sized to accommodate an IUD such that the IUD is slidable along the passageway.

2. The insertion device of claim 1, wherein a maximum straight-line distance between two points on the exterior surface and at the second end of the introducer is greater than 1 cm, more preferably greater than 1.5 cm, more preferably greater than 2 cm, more preferably greater than 2.5 cm, more preferably between 2.5 cm and 4 cm, more preferably between 2.75 cm and 3.25 cm, and most preferably is about 3 cm.

3. The insertion device of claim 1, wherein at least a portion of the exterior surface of the introducer is cylindrical and has a first radius.

4-5. (canceled)

6. The insertion device of claim 3, wherein said at least a portion extends completely or part way to the first end.

7-8. (canceled)

9. The insertion device of claim 1, further comprising a grip portion integrally formed or coupled to the introducer at or proximate to the first end.

10. (canceled)

11. The insertion device of claim 1, wherein the introducer is configured such that at least a portion of the elongate passageway is circular.

12. The insertion device of claim 1, wherein the introducer is configured such that at least a portion of the elongate passageway is non-circular.

13-14. (canceled)

15. The insertion device of claim 1, wherein a cross-sectional area of the space formed by the elongate passageway transitions from a first area at the first end to a second area at a point between the first end and the second end, wherein the first area is greater than the second area.

16. (canceled)

17. The insertion device of claim 1, wherein a cross-sectional area of the space formed by the elongate passageway at the second end is greater than a cross-sectional area of the space formed by the elongate passageway at a point between the first end and the second end.

18. The insertion device of claim 15, wherein a cross-sectional area of the space formed by the elongate passageway transitions from a third area at the second end to a fourth area at a point between the first end and the second end, wherein the third area is greater than the fourth area.

19. (canceled)

20. The insertion device of claim 18, wherein the second area is the same as the fourth area.

21-22. (canceled)

23. The insertion device of claim 1, further comprising one or more orientation features for orientating the pusher and/or the IUD inside the passageway.

24-25. (canceled)

26. The insertion device of claim 1, further comprising a bump joint between the formed between the elongate passageway and the pusher and/or the IUD, said bump temporarily fixing the pusher and/or the IUD in a predetermined position in the passageway until additional force is used to urge the pusher.

27. The insertion device of claim 1, wherein the pusher is configured to be slidably engaged and/or received in the elongate passageway, the pusher comprising a first end and a second end.

28. (canceled)

29. The insertion device of claim 27, wherein the pusher comprises an elongate rod.

30-33. (canceled)

34. The insertion device of claim 27, wherein the pusher comprises a body provided at the first end of the rod, the body configured to engage with an IUD.

35-40. (canceled)

41. The insertion device of claim 27, further comprising a cutout or depression in the pusher that extends longitudinally from a position at or near the first end of the pusher to a position farther from the first end.

42-43. (canceled)

44. The insertion device of claim 27, wherein the pusher comprises one or more indicia to indicate the extent to which the pusher is inserted into the introducer.

45. The insertion device of claim 27, wherein the pusher comprises a mechanical stop configured to engage a corresponding feature of the introducer to limit the extent of insertion of the pusher into the introducer.

46. (canceled)

47. The insertion device of claim 1, further comprising one or more projections on the introducer that extend into the elongate passageway so as to control a position and/or orientation of the pusher and/or IUD in use.

48-49. (canceled)

50. A method of inserting an IUD, comprising:
inserting an IUD into the elongate passageway of the introducer of the insertion device of claim 1 at the first end thereof;
inserting the pusher into the elongate passageway at the first end of the introducer and urging the pusher along the elongate passageway a first distance;
inserting the insertion device into the patient such that the second end of the introducer abuts the fundus;
urging the pusher further into the elongate passageway such that the IUD springs free of the introducer;
retracting the pusher from the introducer; and
retracting the introducer from the patient.
